(19) United States
(12) Patent Application Publication
(10) Pub. No.: US 2025/0153213 A1
(43) Pub. Date: May 15, 2025

(54) FLUID PRODUCT DISPENSER

(71) Applicant: APTAR FRANCE SAS, LE NEUBOURG (FR)

(72) Inventors: Antoine AILLIOT, FERRIÈRES-HAUT-CLOCHER (FR); Denis COURDIER, OYONNAX (FR); Stéphane DAVIOT, GRAVERON SEMERVILLE (FR); Timothée HAQUET, VITOT (FR); Francis MOREAU, SOTTEVILLE LES ROUEN (FR); Emmanuel PEREZ, BOISSEY-LE-CHATEL (FR)

(73) Assignee: APTAR FRANCE SAS, LE NEUBOURG (FR)

(21) Appl. No.: 18/839,086

(22) PCT Filed: Feb. 15, 2023
(86) PCT No.: PCT/FR2023/050203

§ 371 Date: Sep. 4, 2024

(30) Foreign Application Priority Data
Feb. 17, 2022 (FR) FR2201402
Aug. 4, 2022 (FR) FR2208075

(51) Int. Cl. B05C 17/005; A45D 34/00; A45D 34/04; B05C 17/01

(52) U.S. Cl. CPC B05C 17/00593; A45D 34/04; B05C 17/01; A45D 2034/005; A45D 2200/055

(57) ABSTRACT

A dispenser having a reservoir module (R′) havingn a case (RT) for accommodating a fluid product cartridge (C) defining a movable wall (C2) biased by a spring (R4) resting on a bearing (R31); and a dispensing module having an outlet valve for controlling the passage of the fluid product released under pressure from the fluid product cartridge (C). The reservoir module (R′) is removably connected to the dispensing module. The bearing (R31) of the spring (R4) is movable relative to the casing (R1′) between an active position in which the spring (R4) is compressed and a rest position in which the spring (R4) is relaxed, thereby allowing the reservoir module (R′) to be connected to the dispensing module with the bearing (R31) in the rest position and the bearing (R31) subsequently in the active position.

The present invention relates to a fluid product dispenser comprising two separable modules, namely:
a reservoir module comprising a case for accommodating a fluid product cartridge defining a movable wall (for example, a piston) biased by a spring resting on a bearing, so as to move the movable wall in order to push fluid product out of the fluid product cartridge, and
a dispensing module closing the case and comprising an outlet valve controlled by an actuation member for controlling the passage of the fluid product released under pressure from the fluid product cartridge.

The reservoir module is removably connected to the dispensing module, so as to give access to the case and thus be able to extract the fluid product cartridge from the case, in particular in order to replace it with a new cartridge. The favoured field of the present invention is that of cosmetics or pharmacy: the dispenser making it possible, for example, to continuously apply a cream or an ointment to the skin, a mucosa, or the hair.

Replacing the cartridge in this type of dispenser is often a difficult operation, due to it being necessary to reconnect the two modules with the spring which presses on the movable wall of the cartridge. This requires dexterity and force. In addition, the cartridge is generally closed by a lid which is pierced during assembly of the two modules. Improper handling can thus lead to a loss of fluid product.

An aim of the present invention is to overcome the abovementioned disadvantages of the prior art by defining a dispenser in which the assembly of the two modules and the pressurisation of the fluid product are consecutive, and not simultaneous: the pressurisation being performed once the modules have been assembled.

To achieve this aim, the present invention proposes that the bearing of the spring be movable relative to the case between an active position in which the spring is compressed and a rest position in which the spring is relaxed, thus making it possible to connect the reservoir module to the dispensing module with the bearing in the rest position, then to move the bearing in the active position. Thus, the spring does not act on the cartridge during the assembly of the modules: the fluid product stored in the cartridge is at atmospheric pressure. It is only then that the bearing is returned into its active position, thus compressing or arming the spring which will then act again on the movable wall of the cartridge and pressurise the fluid that it contains.

In an embodiment, the spring can be disposed between a thrust part and a bearing part which are slidably movable relative to one other as well as relative to the case, the thrust part forming a thrust head in contact with the movable wall of the fluid cartridge, and the bearing part forming the bearing, the spring acting between the thrust head and the bearing, so as to bias the thrust and bearing parts away from one other. In other words, the thrust and bearing parts slide into one other by compressing/relaxing the spring. The thrust head and the bearing are advantageously located at opposite ends of the thrust and bearing parts, such that the spring extends over the entire length of the two parts.

According to a feature of the invention, the bearing part can be secured to the case in the active position, the thrust part, during the opening of the outlet valve, being movable relative to the case and to the bearing part in the active position. In short, only the thrust part moves in the case to push the movable wall of the cartridge.

Advantageously, the bearing part and the thrust part are in mutual abutment in the rest position with the relaxed spring, the bearing part and the thrust part in mutual abutment being movable relative to the case in the absence of a cartridge full of fluid product. The spring does not need to be totally relaxed: it is sufficient that it is in its most relaxed state, preferably close to its totally relaxed state. The spring can slightly bias the two parts in mutual abutment, or not. When the cartridge has been removed from the case, the assembly formed by the thrust and bearing parts in mutual abutment can slide in the case, at least over a certain stroke.

According to another feature of the invention, the bearing part can be removably snap-fitted onto a hooking profile of the case in the active position. Thus, it is sufficient to push the bearing part fully into the case to perform the snap-fitting. According to a particularly advantageous feature, the thrust part comprises a release profile which acts on the bearing part to release it from its snap-fitting with the case, when the fluid product cartridge is emptied. Preferably, the bearing part forms at least one flexible snap-fitting tab provided with a snap-fitting tooth adapted to snap-fit with the hooking profile of the case, the release profile of the thrust part deforming the flexible snap-fitting tab so as to disengage the snap-fitting tooth from the hooking profile of the case. Advantageously, the spring biases the thrust and bearing parts away from one other, when the release profile acts on the bearing part, such that the bearing part, once released from its snap-fitting with the case, is moved by the spring outside of the case into the rest position, thus giving the user a visual, audible and/or tactile indication that the fluid product cartridge is empty.

According to another aspect of the invention, the thrust part can be moved in the case between a full abutment position and an empty abutment position, corresponding respectively to the full and empty states of the fluid product cartridge. Advantageously, the bearing is in the active position when the thrust part is moved by the spring from its full abutment position to in the proximity of its empty abutment. Preferably, the bearing is moved into the rest position when the thrust part reaches its empty abutment position.

Moreover, the outlet valve is advantageously biased into a closed state by the pressurised fluid product.

In another advantageous embodiment, the reservoir module can further comprise a planet gear, which engages with both the case and the bearing part to switch between the active and rest positions. Preferably, the planet gear is trapped in the bearing part with a limited axial movement and a rotational movement, the planet gear selectively stably engaged with the case in the active position. Moreover, the bearing part can comprise at least one rotary drive cam for rotatably biasing the planet gear. Advantageously, the case can comprise at least one locking cam and one ejection cam, the planet gear comprising at least one lug which slides over the locking and ejection cams under the action of the spring, which acts on the planet gear through the bearing part. The case can form an axial abutment, the planet gear thus being biased against this axial abutment by the bearing part.

According to a practical embodiment,
the case can comprise at least one locking cam and one ejection cam connected by a stop wall, an axial abutment and at least one axial funnel,
the bearing part can comprise an axial stroke-limiting abutment and at least one rotary drive cam,
the planet gear can comprise at least one lug, which defines a sliding surface, an abutment ring and teeth.

In this case, the teeth engage the rotary drive cam to rotatably bias the planet gear during the compression phases of the spring. The abutment ring engages with the axial stroke-limiting abutment during the relaxation phases of the spring. The lug moves in the axial funnel and between the locking and ejection cams and the axial abutment under the biasing of the bearing part. The sliding surface comes into sliding contact on the locking and ejection cams. The lug comes into contact with both the locking cam and the stop wall in the active position. The rotary drive cam rotates the planet gear when the lug is in contact with the axial abutment. Thus, the complete cycle of the lug is as follows: the lug is first moved axially in the axial funnel by pressing on the bearing part until it comes into contact with the axial abutment: the lug is thus rotatably moved under the action of the rotary drive cam. The lug then comes into contact with the locking cam when the bearing part is released: the lug slides over the locking cam until it comes into contact with the stop wall, marking the active position. The lug then slides against the stop wall by pressing on the bearing part until it comes into contact with the axial abutment: the lug is thus rotatably moved under the action of the rotary drive cam. The lug thus comes into contact with the ejection cam when the bearing part is released. The lug finally slides over the ejection cam until it falls into another axial funnel.

This mechanism and this operation is similar to those pens with a retractable tip by pressing an end button. A first press on the button followed by a release makes it possible to take the lead out of the pen and lock it in the active writing position. A second press followed by a release makes it possible to retract the lead. This same principle is used in the present invention to arm/disarm the spring. Starting from the active position, a first press on the bearing part followed by a release makes it possible to disarm the spring and a second press followed by a release makes it possible to arm the spring again.

The invention also defines a method for loading a dispenser such as defined above, comprising the following successive steps:
a) disposing the bearing in the rest position,
b) disconnecting the reservoir module from the dispensing module,
c) introducing a fluid cartridge into the reservoir module,
d) connecting the reservoir module to the dispensing module,
e) moving the bearing into the active position.

Steps a) and b) can optionally be reversed: the main thing being that the connection is made with the bearing in the rest position.

Advantageously, the bearing automatically returns into the rest position when the fluid product cartridge is emptied. Step a) is therefore automatic and does not need the intervention of the user. It has been seen previously that the thrust part, when it reaches the end of its stroke, acts on the bearing part to release it from its snap-fitting with the case.

The spirit of the present invention is based on disarming the spring so as to be able to connect the two modules without being impeded by the pressure exerted by the spring. This disarming can result from a handling of the user, but preferably it is automatically triggered when the cartridge has been emptied of its contents. Thus, the user does not have to be concerned about disarming the spring and further receives a visual, audible and/or tactile indication that the cartridge is empty. Indeed, the release of the bearing can naturally be seen, since it moves relative to the case, but it can also generate a sound and/or a small impact in the dispenser that the user can feel in their hand.

The invention will now be more fully described in reference to the accompanying drawings, giving as a non-limiting example, an embodiment of the invention.

In the figures:

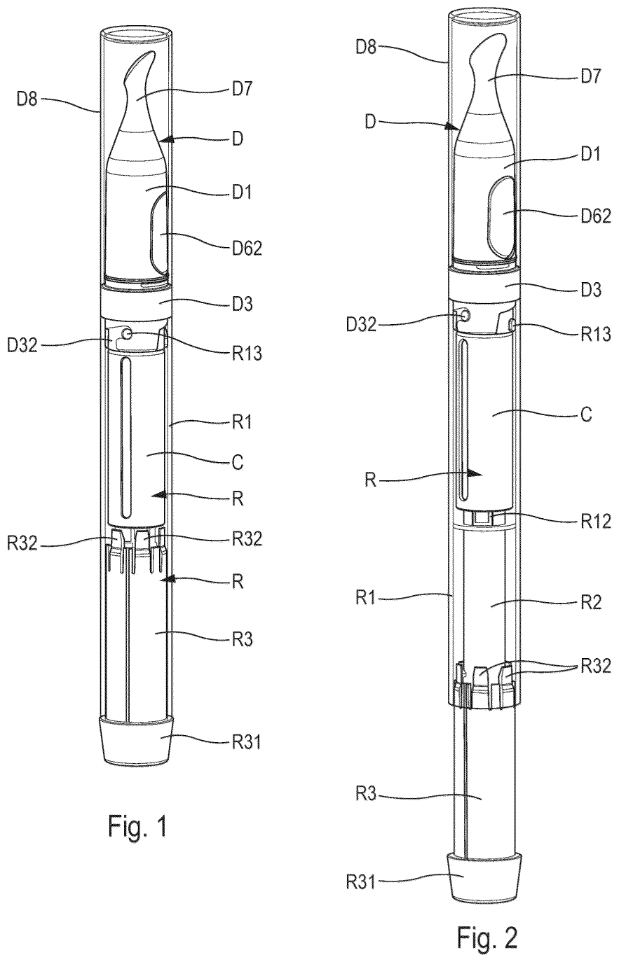

FIG. 1 is a perspective and partially transparent view of a fluid product dispenser of the invention in an operating state,

FIG. 2 is a view similar to that of FIG. 1 with the dispenser in a rest state,

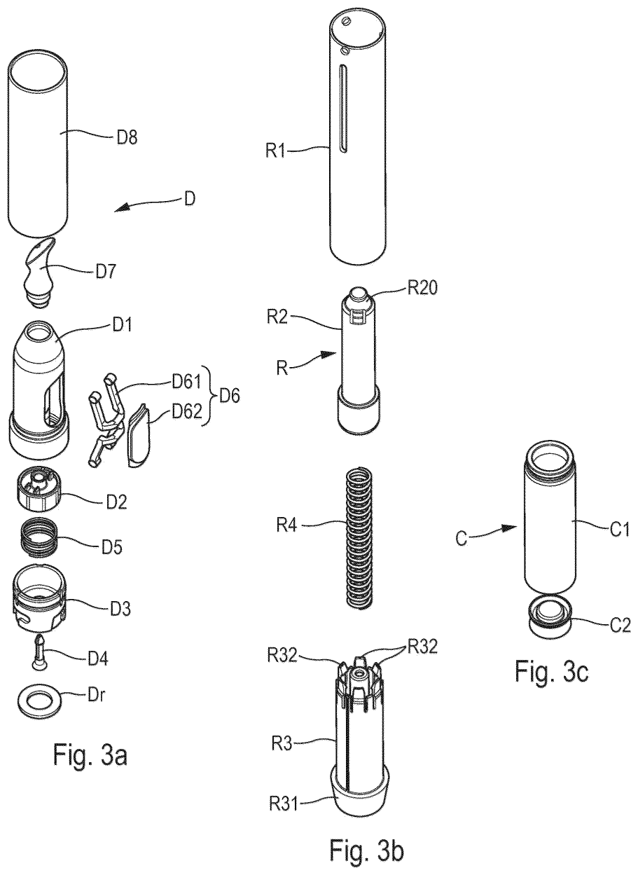

FIG. 3a is an exploded, perspective view of the dispensing module of the dispenser of FIGS. 1 and 2,

FIG. 3b is an exploded, perspective view of the reservoir module of the dispenser of FIG. 1,

FIG. 3c is an exploded, perspective view of the fluid product cartridge of the dispenser of FIGS. 1 and 2,

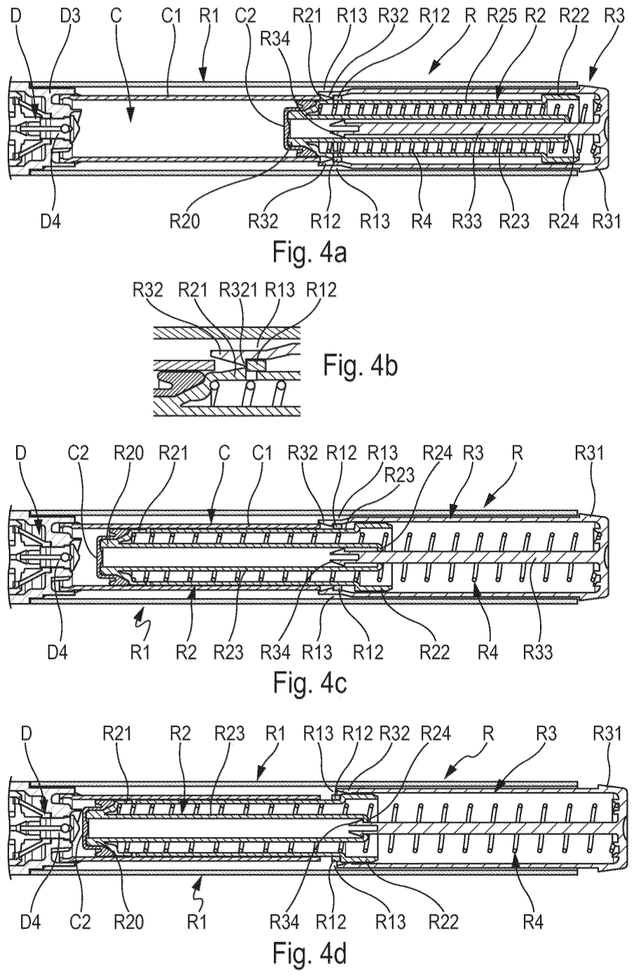

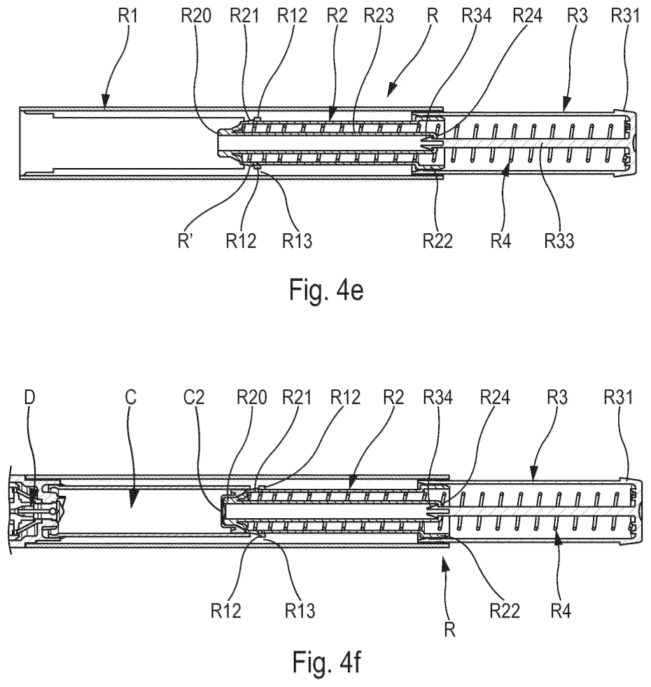

FIGS. 4a, 4c, 4d, 4e and 4f are longitudinal cross-sectional views through the reservoir module of the dispenser of the invention under different conditions to illustrate the operation of this reservoir module,

FIG. 4b is a greatly enlarged view of a detail of FIG. 4a,

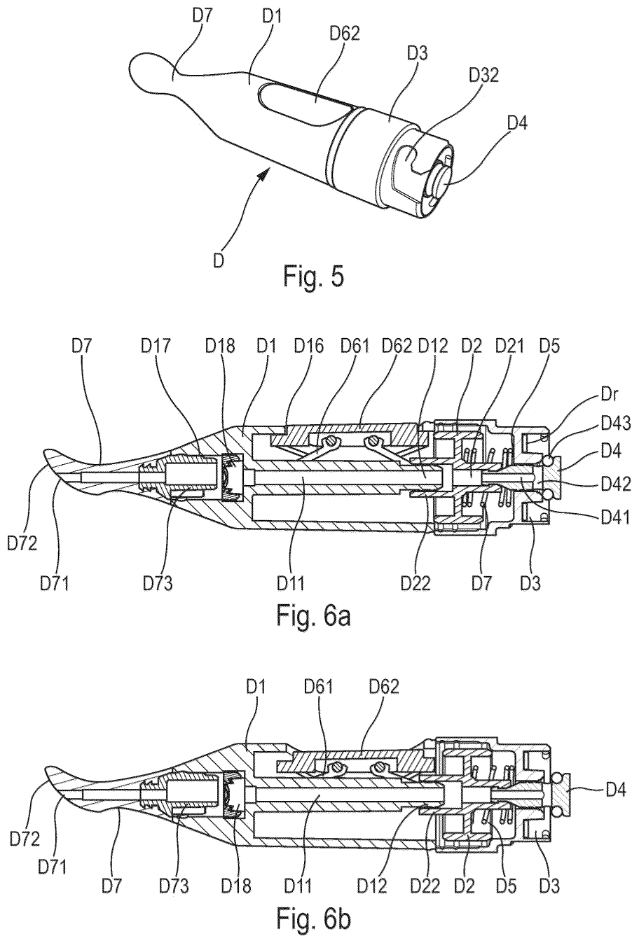

FIG. 5 is a perspective view of the dispensing module of the dispenser of the invention,

FIGS. 6a and 6b are longitudinal, cross-sectional views through the dispensing module of FIG. 5, respectively in the rest position and in the actuated position, and

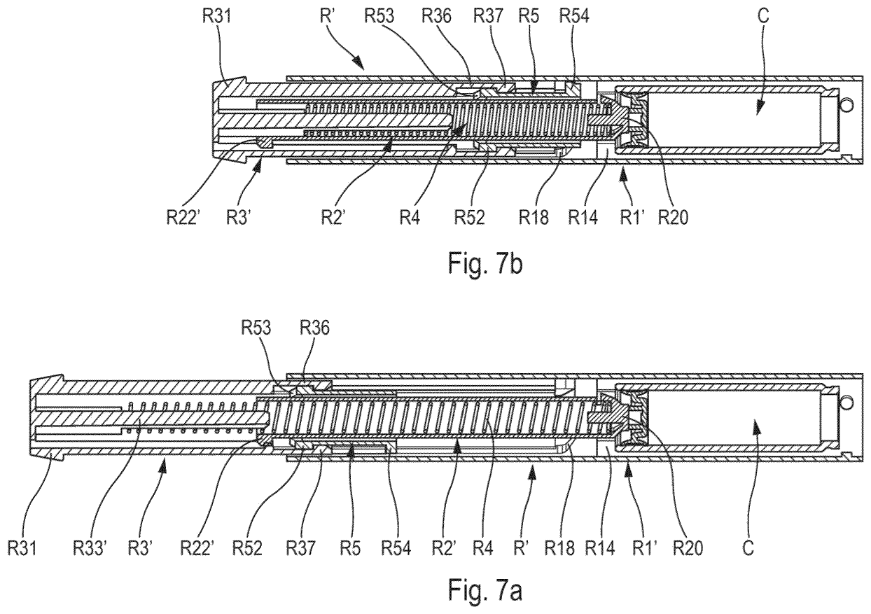

FIGS. 7a and 7b are cross-sectional views through the dispenser according to a second embodiment of the invention, respectively in the rest and operating position,

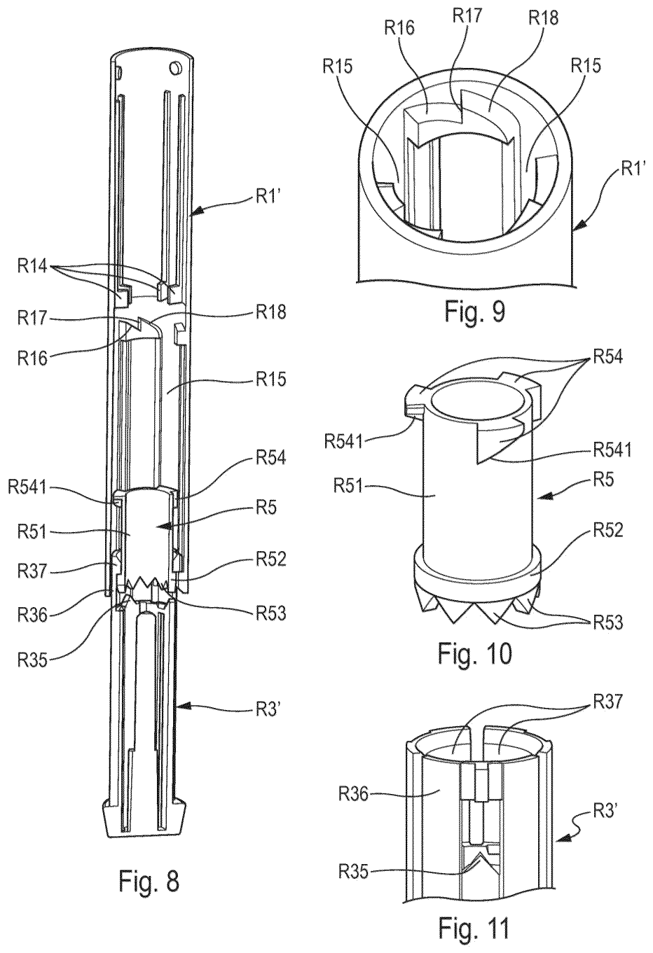

FIG. 8 is a perspective, cropped view showing the inside of the dispenser,

FIGS. 9, 10 and 11 are enlarged, perspective views of the parts which can be seen in FIG. 8, and

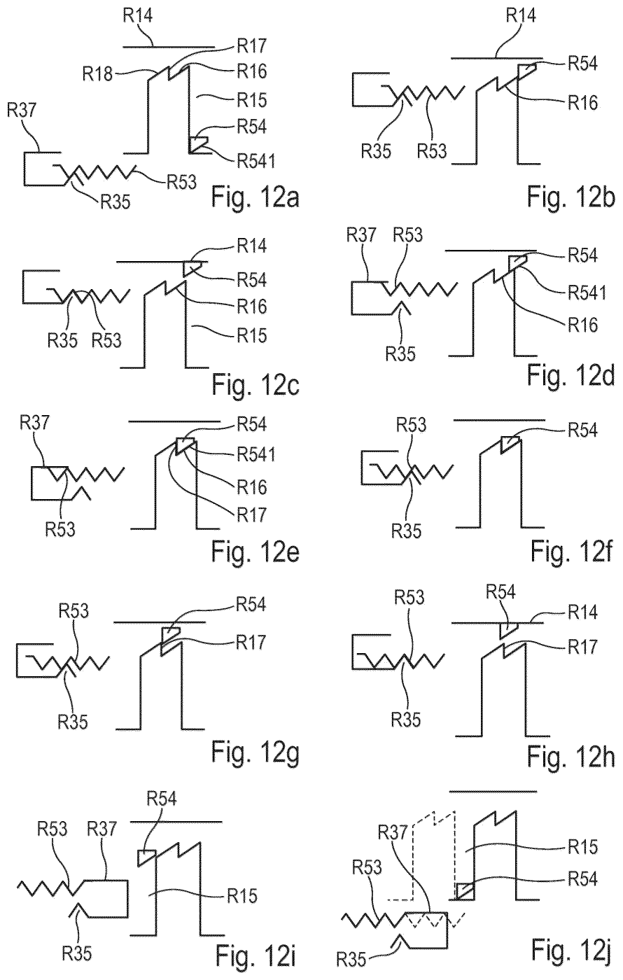

FIGS. 12a to 12j are diagrams illustrating the different steps of the operation of the dispenser of FIGS. 7a to 11.

The fluid product dispenser which has been used to illustrate the present invention is of a particular type, since this is an applicator comprising an application head D7 making it possible not only to dispense the fluid product, but also to apply it to the desired target surface, which can be the skin, nails, hair, etc. It must be understood that the invention is not limited to this particular type of dispenser/applicator, but that it applies to any type of dispenser.

The dispenser of the invention comprises two distinct sub-assemblies that can be removably connected together, namely a reservoir module R and a dispensing module D, which can be assembled and separated by means of a removable or reversible connection, such as a screw connection, a snap-fitting connection, a bayonet connection, etc. In the dispenser illustrating the invention, the removable connection is a bayonet connection implementing a suitable housing D32 for accommodating a lug R13. Thus, by relative rotation between the reservoir module R and the dispensing module D, the lug R13 can be inserted/extracted from the housing D32. It is possible to provide several housings D32 and several lugs R13 for a balanced connection.

Generally, the reservoir module R comprises a case R1 intended to removably accommodate a fluid product cartridge C. To bias this cartridge C, the reservoir module R comprises a thrust part R2 which engages with a bearing part R3. Although not visible in FIGS. 1 and 2, a spring acts between the thrust part R2 and the bearing part R3. It can be seen in FIGS. 1 and 2 that the bearing part R3 comprises a bearing R31, as well as flexible snap-fitting tabs R32. The reservoir module R will be more fully described below.

The dispensing module D, such as can be seen in FIGS. 1 and 2, comprises a body D1, at the upper end of which the application dispensing head D7 is mounted. At its opposite end, the dispensing module D comprises a base D3 secured to the body D1 and forming the housing D32. The body D1 forms a window in which a pushbutton D62 is disposed. Optionally, the dispensing module D comprises a removable protective cap D8, which engages with the body D1 or the base D3.

By referring to FIG. 3a, all the component parts of the dispensing module D can be seen. It must be borne in mind that this is only one non-limiting embodiment. The dispensing module D comprises the body D1, the application dispensing head D7, the base D3, the protective cap D8, as well as a shuttle D2 supporting an outlet valve D4, a spring D5, a flat seal Dr, a toggle D61 and a pushbutton D62 together forming an actuation member D6. The toggle D61 can be made of one part or of two parts. It can even be made of one piece with the pushbutton D62.

In FIG. 5, the dispensing module D in perspective in the mounted state can be seen. The body D1, the application dispensing head D7, the pushbutton D62, the base D3 with its bayonet housing D32, as well as a part of the outlet valve D4 can be distinguished.

The operation of this dispensing module D is more understandable from FIGS. 6a and 6b. The body D1 comprises a side window D16, in which the pushbutton D62 is housed. The body D1 is passed through by a conduit D11 which defines a sliding endpiece D12 upstream and comprises a closing valve D18 downstream. Further downstream, the body D1 comprises an anchoring housing D17 for an anchoring bead D73 forming part of the application dispensing head D7. It can also be noted that the head D7 is passed through by a channel which ends by a dispensing orifice D71 at an application surface D72. It can be said that the sliding endpiece D12 is fluidically connected to the dispensing orifice D71 by passing through the valve D18 and the anchoring stub D73.

The base D3 is fixedly mounted on the body D1 as mentioned above: the base D3 forms one or more bayonet housing(s) D32. The base D3 also contains the shuttle D2, which comprises a sliding sleeve D22 sealingly and slidingly engaged on the sliding endpiece D12 of the body D1. The shuttle D2 internally defines a passage D21 which communicates with the conduit D11. The outlet valve D4 is fixedly mounted on the shuttle D2 and internally defines a valve channel D41, which is extended by side outlets D42. The outlet valve D4 also comprises a sealing element D43, which can be presented in the form of an O-ring which sealingly bears on the base D3. The sealing element can also be formed on the base D3. The spring D5 bears on the base D3 and biases the shuttle D2 towards the body D1, i.e. in a direction where the sealed sliding fit between the sleeve D22 and the endpiece D12 is at a maximum. This is represented in FIG. 6a, which corresponds to the rest position of the dispensing module D. It can be noted that the seal Dr is mounted in the base D3.

The actuation member D6, which comprises the toggle D61 and the pushbutton D62, acts between the body D1 and the shuttle D2 so as to move the shuttle D2 away from the body D1, so as to detach the sealing element D43 from the base D3. This is represented in FIG. 6b. To do this, the toggle D61 bears, on the one hand, on the body D1 and, on the other hand, on the shuttle D2. By laterally pressing on the pushbutton D62, the toggle D61 is deformed, which causes the shuttle D2 to move to the right in FIGS. 6a and 6b, i.e. away from the body D1. This movement is made against the force exerted by the spring D5. Thus, the toggle D61 constitutes a means making it possible to transform a radial force into an axial force acting between the body D1 and the shuttle D2 to control the opening of the outlet valve D4.

This design for the dispensing module D is advantageous, but not unique: other designs can be considered, insofar as the dispensing module D comprises a controllable outlet valve, and can be removably connected to the reservoir module R.

FIG. 3c shows the fluid product cartridge C. This comprises a barrel C1 in which a piston C2 is engaged, which sealingly slides inside the barrel C1 to decrease its useful volume. Instead of the piston, a flexible pocket can also be used. Generally, the cartridge C must comprise a movable wall, whether it is sliding like the piston C2 or deformable like a pocket, in order to define a reservoir of variable volume. The cartridge C filled with fluid product can be closed by a pierceable or peelable lid, not represented.

FIG. 3b shows the component elements of the reservoir module R, namely the case R1, the thrust part R2, the bearing part R3 and the spring R4. It can be seen that the bearing module R3 comprises the bearing R31, as well as flexible snap-fitting lugs R32 at the opposite end. It can be distinguished that the flexible tabs R32 form inward-oriented snap-fitting teeth R321. The thrust R2 and bearing R3 parts are intended to be assembled so as to contain the spring R4 which, on the one hand, bears on the bearing R31 of the bearing part 3 and, on the other hand, pushes the thrust head R20 of the thrust part R2 against the piston C2 of the fluid product cartridge C, thus bringing it to sealingly slide inside the barrel C1.

Reference will now be made to FIG. 4a which shows the reservoir module R in its operating state, corresponding to FIG. 1. In the left part of FIG. 4a, the lower part of the dispensing module D can be seen. The case R1 of the reservoir module R is closed by the dispensing module D, as mentioned above, i.e. by a removable bayonet fastening. It can also be noted that the barrel C1 is sealingly engaged with the base D3. Thus, the content of the cartridge C communicates directly with the outlet valve D4. The lid of the cartridge C has, for example, been broken by a suitable profile formed by the base D3.

According to the invention, the case R1 internally forms several profiles R12, which can, for example, be in the form of strips formed of one piece with the case R1, but separated from the internal wall of the case R1 by an access passage R13. This can be seen more clearly in FIG. 4b. These strips can be rectilinear or curved: their two ends being connected to the internal wall of the case R1. These profiles (or strips) R12 are located substantially in the middle of the case R1 and are distributed along an annular line. It must be noted that the flexible snap-fitting tabs R32 of the thrust part R3 are engaged through the access passages R13 such that their snap-fitting teeth R321 snap-fittingly engage with the profiles R12. It must also be noted that the thrust part R2 forms elastic blades R21 which abut against the profiles R12. It can thus be said that the elastic blades R21 engage with the profiles R12 on the same face of the profiles as the snap-fitting teeth R321. In this active position, the spring R4 is maximally compressed inside the assembly formed by the thrust part R2b and the bearing part R3. The spring R4 bears on the bearing R31 of the bearing part R3 and acts directly on the thrust head R20 of the thrust part R2. The spring R4 is mainly engaged inside the thrust part R2 which forms a cylinder R25 which ends by a shoulder R22 of increased diameter. The thrust part R2 also comprises an internal tube R23 which ends by a passage opening R24. The bearing part R3 extends around the cylinder R25 from its bearing 31 to its flexible snap-fitting tabs R32. Internally, the bearing part R3 comprises a rod R33 engaged inside the tube R23 through the passage opening R24. This rod R33 ends by one or more hooking profile(s) R34, which enable the insertion of the rod R33 in the tube R23, but which prevent it from being extracted by abutting the hooking profiles R34 on the edge of the passage opening R24, as will be seen below. Other means for associating the rod R3 and the tube R23 are also possible.

Thus, in this active position, the bearing part R3 is retained in the case R1 by the passage of the flexible tabs R32 through the access passages R13 and their snap-fitting on the profiles (strips) R12: the case R31 practically abutting against the end of the case R1. It can be said that the bearing R31 is fixed relative to the case R1 in this active position. The spring R4, which is compressed to the maximum, therefore acts between the bearing R31 and the thrust head R20 of the thrust part R2, which is in direct contact with the piston C2 of the cartridge C. The fluid product contained in the cartridge C is thus pressurised. The fact that the outlet valve D4 is closed, cannot however be escaped from.

By pressing on the pushbutton D62, the outlet valve D4 opens and the pressurised fluid product in the cartridge C is pushed through the dispensing module D until it reaches its dispensing orifice D71. The dispensing of the fluid product is accompanied by a movement of the piston C2 under the action of the thrust head R20, which is biased by the spring R4 which rests on the fixed bearing R31 of the bearing part R3. The thrust part R2 moves inside the barrel C1, except for its shoulder R22 which slides inside the bearing part R3. The movement of the shoulder R22 in the bearing part R3 can be performed by sliding with or without contact. It can be noted in FIG. 4c that the shoulder R22 moves closer to the flexible snap-fitting tabs R32, which are engaged with the profiles R12. The rod R33 is out of the tube R23, but the hooking profiles R34 are located inside the tube R23. The cartridge C is almost empty, since the piston C2 arrives in the proximity of the outlet valve D4.

In FIG. 4d, the cartridge C is now empty: the piston C2 being in the proximity of the outlet valve D4. Above all, it must be noted that the shoulder R22 of the thrust part R2 is now located at the flexible snap-fitting tabs R32, which are biased outwards by the shoulder R22, so as to be disengaged from their snap-fitting with the profiles R12 of the case R1. The shoulder R22 is thus abutted against the profiles R12, marking the end of the stroke of the thrust part R2, and thus, due to the piston C2. Under the effect of the spring R4, the bearing part R3 has been moved to the right, leading to the flexible snap-fitting tabs R32 being moved or extracted from the access passages R13 and the bearing 31 being moved away from the end of the case R1. The spring R4 is thus in its rest position, relaxed or almost relaxed. This rest position is fixed by the engagement profiles R34 bearing against the edge of the passage opening R24.

Subsequently, the assembly consisting of the thrust part R2 and the bearing part R3 is free to slide inside the case R1. This unitary assembly can therefore be moved into the position of FIG. 4e, where it is noted that the dispensing module D has been removed from the case R1 and that the empty cartridge C has been extracted from the same case R1. In this open rest position, the elastic blades R21 have been able to be relaxed out of the cartridge C to abut against one face of the profiles R12. Naturally, the hooking profiles R34 are always abutted against the edge of the passage opening R24. The spring R4 is relaxed or almost relaxed. In any case, it is in its most relaxed state.

It is thus understood that it is easy to introduce a new filled cartridge C inside the case R1, until its piston C2 abuts against the thrust head R20 of the thrust head R2. The dispensing module D can thus be replaced on the reservoir module R. Advantageously, the base D3 can form a piercing profile to pierce or cut the lid which seals the cartridge C. It is thus in the configuration represented in FIG. 4f. It must be noted that the thrust head R20 does not bias the piston C2, given that the spring R4 is relaxed. To arm the dispenser again, it is sufficient to push using a finger on the bearing R31 so as to slide the bearing part R3 inside the case R1 around the thrust part R2. The sliding of the bearing part R3 has the effect of compressing the spring R4 and of biasing the thrust head R20 against the piston C2. The bearing part R3 arrives in its final active position, when the flexible snap-fitting tabs R32 again snap-fittingly engage on the profiles R12, after passing through the access openings R13. This final active position is that of FIG. 4a, where it can be seen that the bearing R31 is abutted again, (or almost) against the end of the case R1.

Through this complete description of an operating cycle of the dispenser, it can be seen that the movement of the bearing R31 relative to the case R1 makes it possible to release the pressure exerted by the spring R4, which enables the insertion of a new cartridge C into the case R1 without undergoing the thrust of the thrust head R20. In other words, the present invention makes it possible to disarm the spring R4 by moving its bearing R31. Once the cartridge is inserted into the case R1 and the dispensing module D is reconnected to the reservoir module R, the spring R4 can again be armed by moving and locking the bearing R31 in its active starting position.

It must also be noted that disarming of the spring R4 occurs automatically when the cartridge C is emptied of its contents, such that the user does not need to act or handle the dispenser to disarm the spring R4. Indeed, it is the thrust part R2 which acts on the bearing part R3 at the end of the stroke to undo the thrust part R3 from its engagement with the case R1. This is a particularly advantageous feature in terms of movement.

The profiles or strips R12 of the case R1 fulfil several functions, namely as a snap-fitting ridge for the flexible tabs R32 and as an abutment surface for the elastic blades R21 and the shoulder R22.

The shoulder R22 making it possible to release the flexible snap-fitting tabs of the profiles R12 fulfil a release function and can therefore be qualified as release profiles.

The thrust part R2 is trapped in the case R1, while sliding between two extreme abutments, namely a full abutment, in which the elastic blades R21 are abutted against the profiles R12, corresponding to a full state of the cartridge C, and an empty abutment, in which the shoulder R22 is abutted against the profiles R12, corresponding to an empty state of the cartridge C. The bearing R3 is in the active position when the thrust part R2 is moved by the spring R4 from its full abutment to the proximity of its empty abutment. The bearing R31 is moved into the rest position when the thrust part R2 reaches its empty abutment.

By referring now to FIGS. 7a, 7b, 8, 9, 10 and 11, a second embodiment can be seen, which differs from the first embodiment, by the mechanism for arming and disarming the spring R4. The dispensing module D, with its outlet valve D4 controlled by the actuation member D6, can be identical or similar to that of the first embodiment. The cartridge C can also be identical or similar.

In this second embodiment, the reservoir module R is distinguished by the implementation of a planet gear R5, which acts between the bearing part R3′ and the case R1′.

More specifically, the case R1′, comparable to the case R1, comprises at least one locking cam R16 and one ejection cam R18 connected by a stop wall R17. The cams R16 and R18 project inside the case R1′ and are curved and inclined. The stop wall R17 is vertical and radial: it extends in a vertical radial plane. The case R1′ also comprises an axial abutment R14, which is defined by several radial vertical flaps, which extend inside the case R1′. The axial abutment R14 is located above the cams R16 and R18, by defining a clearance space between them. The case R1′ also defines at least one axial funnel R15. As can be seen in FIG. 9, the internal wall of the case R1′ forms three identical assemblies, each comprising a locking cam R16, a stop wall R17, an ejection cam R18 and an axial funnel R15.

The bearing part R3′, comparable to the case R3, comprises three rotary drive cams R35, which, in this case, are presented in the form of substantially triangular teeth which point in the direction of the cartridge C. The bearing part R3′ also comprises three tabs R37, which are slightly deformable. The three rotary drive cams R35 are disposed between the three tabs R37. The three tabs R37 together form an axial stroke-limiting abutment R38, in the form of an inner reinforcement defining an annular shoulder. The spring R4 is not represented, but it bears on the bottom of the bearing part R3′, like in the first embodiment in R31.

The planet gear R5 is a one-piece part which comprises a substantially cylindrical body R51. As can be seen in FIG. 10, the planet gear R5 comprises or forms three lugs R54 at its upper end. These lugs R54 project outwards from the body R51 and each define an inclined sliding surface R541. At its opposite lower end, the body forms an abutment ring R52 and teeth R53, which project outwards from the body R51. The teeth R53 can be adjacent and extend over the entire periphery of the lower edge of the body R51. The teeth R53 are substantially triangular-shaped and point in the direction of the teeth of the rotary drive cams R35.

The case R1′, the bearing part R3′ and the planet gear R5 engage as follows. The teeth R53 of the planet gear R5 engage with the teeth of the rotary drive cams to rotatably bias the planet gear R5 during the compression phases of the spring R4, i.e. when the user presses on the bearing part R3′ against the spring R4. The abutment ring R52 engages with the axial stroke-limiting abutment R37 during the relaxation phases of the spring R4, i.e. when the user relaxes their pushing on the bearing part R3′. The lugs R54 move in the axial funnels R15 and in the clearance space defined between the locking R16 and ejection R18 cams and the axial abutment R14, under the biasing of the bearing part R3′. The sliding surface R541 of the lugs R54 comes into sliding contact on the locking R16 and ejection R18 cams: the lugs coming into contact both with the locking cams R16 and the stop walls R17 in the active position. The teeth of the rotary drive cams R35 rotate the planet gear R5 when the lugs R54 are in contact with the axial abutment R14.

By referring to FIGS. 12a to 12j, a complete cycle of the path of a lug R54 is described.

In FIG. 12a, the lug R45 is at the bottom of the axial funnel R15. This corresponds to the disarmed rest position of FIG. 7a.

In FIG. 12b, the user presses on the bearing part R3′, such that the tooth of the rotary drive cam R35 is engaged between two teeth R53 of the planet gear, but cannot fully engage between these two teeth, due to the axial guiding of the lug R54 in the axial funnel R15. The lug R54 therefore moves axially in the axial funnel R15, while being rotatably biased, but without being able to rotate.

In FIG. 12c, the lug R54 has been released from the axial guiding of the funnel R15 and has come into contact with the axial abutment R14: the lug R54 is thus rotatably moved under the action of the rotary drive cam R35, the tooth of which is thus fully engaged between the two teeth R53 of the planet gear. It can be noted that the lug R54 is located partially above the locking cam R16.

In FIG. 12d, the user has relaxed their pressure on the bearing part R3′, such that the tooth of the rotary drive cam R35 has been disengaged from the two teeth R53 of the planet gear, which are now abutted against the axial stroke-limiting abutment R38. Under the biasing of the abutment R38, subjected to the action of the spring R4, the sliding surface R541 of the lug R54 is lowered so as to catch the locking cam R16.

In FIG. 12e, the lug R54 has slid on the locking cam R16 until it comes into contact with the stop wall R17, corresponding to the armed active position. This position is stable, since the lug R54 is biased by the spring R4, which acts on the abutment R38 which is in contact with the teeth R53 of the planet gear.

In FIG. 12f, the user presses on the bearing part R3′, such that the tooth of the rotary drive cam R35 has engagingly returned between two teeth R53 of the planet gear, but cannot fully engage between these two teeth, because of the axial guiding of the stop wall R17.

In FIG. 12g, the lug R54 slides against the stop wall R17.

In FIG. 12h, the lug R54 is released from the stop wall R17 and comes into contact with the axial abutment R14. The lug R54 has rotatably moved under the action of the rotary drive cam R35: its tooth is again fully engaged between the two teeth R53 of the planet gear.

In FIG. 12i, the user has relaxed their pressure on the bearing part R3′, such that the tooth of the rotary drive cam R35 has been disengaged from the two teeth R53 of the planet gear, which are now abutted against the axial stroke-limiting abutment R38. Under the biasing of the abutment R38, subjected to the action of the spring R4, the sliding surface R541 of the lug R54 is lowered so as to slide on the ejection cam R18.

In FIG. 12j, the lug R54 has left the ejection cam R18 and has fallen into another axial funnel R15. The disarmed rest position is returned into.

This type of mechanism with a planet gear biased in axial and rotary movement by cams and abutments is known per se, but the present invention implements it in a particular application, in which it serves to arm/disarm a spring to allow an easy replacement of the dispensing cartridge.

The dispensers of the invention make it possible to define a loading method, comprising the following successive steps:
a) disposing the bearing in the rest position,
b) disconnecting the reservoir module from the dispensing module,
c) inserting a fluid product cartridge into the reservoir module,
d) connecting the reservoir module to the dispensing module,
e) moving the bearing into the active position.

Thanks to the invention, a spring-loaded reservoir module, the replacement of a fluid product cartridge of which is greatly facilitated by the absence of force exerted by the spring during the reconnection of the dispensing module.

1. Fluid product dispenser comprising:
a—a reservoir module comprising a case accommodating a fluid product cartridge defining a movable wall biased by a spring resting on a bearing, so as to move the movable wall to push fluid product out of the fluid product cartridge,
b—a dispensing module closing the case and comprising an outlet valve controlled by an actuation member for controlling the passage of the fluid product released under pressure from the fluid product cartridge,
wherein the reservoir module is removably connected to the dispensing module, so as to give access to the case (R1; R1′) and thus be able to extract the fluid product cartridge from the case,
characterised in that the bearing of the spring is movable relative to the case between an active position in which the spring is compressed and a rest position in which the spring is relaxed, thus making it possible to connect the reservoir module to the dispensing module with the bearing in the rest position, then to move the bearing in the active position.

2. Dispenser according to claim 1, wherein the spring is disposed between a thrust part and a bearing part which are slidably movable relative to one another as well as relative to the case, the thrust part forming a thrust head in contact with the movable wall of the fluid product cartridge and the bearing part forming the bearing, the spring acting between the thrust head and the bearing, so as to bias the thrust parts and the bearing parts away from one another.

3. Dispenser according to claim 2, wherein the bearing part is secured to the case in the active position, the thrust part, when the outlet valve is opened, being movable relative to the case and to the bearing part in the active position.

4. Dispenser according to claim 2, wherein the bearing part and the thrust part are in mutual abutment in the rest position with the relaxed spring, the bearing part and the thrust part in mutual abutment being movable relative to the case in the absence of a full fluid product cartridge.

5. Dispenser according to claim 2, wherein the bearing part is removably snap-fitted to a hooking profile of the case in the active position.

6. Dispenser according to claim 5, wherein the thrust part comprises a release profile which acts on the bearing part to release it from snap-fitting with the case, when the fluid product cartridge is emptied.

7. Dispenser according to claim 6, wherein the bearing part forms at least one flexible tab provided with a snap-fitting tooth adapted to snap-fittingly engage with the hooking profile of the case, the release profile of the thrust part deforming the flexible tab so as to disengage the snap-fitting tooth of the hooking profile from the case.

8. Dispenser according to claim 6, wherein the spring biases the thrust and bearing parts away from one other, when the release profile acts on the bearing part, such that the bearing part, once released from its snap-fitting with the case, is moved by the spring out of the case into the rest position, thus giving the user a visual, audible and/or tactile indication that the fluid product cartridge is empty.

9. Dispenser according to claim 2, wherein the thrust part is movable in the case between a full abutment and an empty abutment, corresponding respectively to the full and empty states of the fluid product cartridge.

10. Dispenser according to claim 9, wherein the bearing is in the active position when the thrust part is moved by the spring from its full abutment to the proximity of its empty abutment.

11. Dispenser according to claim 10, wherein the bearing is moved into the rest position when the thrust part reaches its empty abutment.

12. Dispenser according to claim 2, wherein the reservoir module further comprises a planet gear which engages with both the case and the bearing part to switch between the active and rest positions.

13. Dispenser according to claim 12, wherein the planet gear is trapped in the bearing part with a limited axial movement and a rotary movement, the planet gear being selectively stably engaged with the case in the active position.

14. Dispenser according to claim 12, wherein the bearing part comprises at least one rotary drive cam for rotatably biasing the planet gear.

15. Dispenser according to claim 12, wherein the case comprises at least one locking cam and one ejection cam, the planet gear comprising at least one lug which slides over the locking and ejection cams under the action of the spring which acts on the planet gear through the bearing part.

16. Dispenser according to claim 12, wherein the case forms an axial abutment, the planet gear being biased against this axial abutment by the bearing part.

17. Dispenser according to claim 12, wherein:
the case comprises at least one locking cam and one ejection cam connected by a stop wall, an axial abutment and at least one axial funnel,
the bearing part comprises an axial stroke-limiting abutment and at least one rotary drive cam,
the planet gear comprises at least one lug which defines a sliding surface, an abutment ring and teeth,
the teeth engaging with the rotary drive cam to rotatably bias the planet gear during the compression phases of the spring, the abutment ring engaging with the axial stroke-limiting abutment during the relaxation phases of the spring, the lug moving in the axial funnel and between the locking and ejection cams and the axial abutment under the biasing of the bearing part, the sliding surface coming into sliding contact on the locking and ejection cams, the lug coming into contact both with the locking cam and the stop wall in the active position, the rotary drive cam rotating the planet gear when the lug is in contact with the axial abutment, such that the lug is moved axially in the axial funnel by pressing on the bearing part until coming into contact with the axial abutment, the lug is thus rotatably moved under the action of the rotary drive cam, the lug coming into contact with the locking cam when the bearing part is released, the lug sliding over the locking cam until coming into contact with the stop wall, marking the active position, the lug sliding against the stop wall by pressing on the bearing part until coming into contact with the axial abutment, the lug is thus rotatably moved under the action of the rotary drive cam, the lug coming into contact with the ejection cam when the bearing part is released, the lug sliding over the ejection cam until falling into another axial funnel.

18. Method for loading a dispenser according to claim 1, comprising the following successive steps:
a) disposing the bearing in the rest position,
b) disconnecting the reservoir module from the dispensing module,
c) introducing a fluid product cartridge into the reservoir module,
d) connecting the reservoir module to the dispensing module,
e) moving the bearing into the active position.
